(19) United States
(12) Patent Application Publication
(10) Pub. No.: US 2024/0075035 A1
(43) Pub. Date: Mar. 7, 2024

(54) METHODS AND COMPOSITIONS FOR TREATING SLEEP APNEA

(71) Applicant: APNIMED, INC. (DELAWARE), Cambridge, MA (US)

(72) Inventors: Ronald FARKAS, Clarksville, MD (US); Luigi TARANTO-MONTEMURRO, Brookline, MA (US); Lawrence G MILLER, Cambridge, MA (US)

(21) Appl. No.: 18/271,716

(22) PCT Filed: Jan. 12, 2022
(86) PCT No.: PCT/US2022/012067

§ 371 Date: Jul. 11, 2023

Related U.S. Application Data
(60) Provisional application No. 63/137,211, filed on Jan. 14, 2021.

(51) Int. Cl. A61K 31/513; A61K 31/138; A61K 31/216; A61P 11/04; A61P 25/20

(52) U.S. Cl. CPC A61K 31/513; A61K 31/138; A61K 31/216; A61P 11/04; A61P 25/20

(57) ABSTRACT

Pharmaceutical compositions comprising Lemborexant or a pharmaceutically acceptable salt thereof and a norepinephrine reuptake inhibitor (NRI) and methods of treating Sleep Apnea comprising administering Lemborexant or a pharmaceutically acceptable salt thereof and an NRI are described herein. In some embodiments, the NRI is atomoxetine or a pharmaceutically acceptable salt thereof. In some embodiments, the pharmaceutical compositions further comprise a muscarinic receptor antagonist (MRA). In some embodiments, the methods of treating Sleep Apnea further comprise administering an MRA. In some embodiments, the MRA is oxybutynin or a pharmaceutically acceptable salt thereof.

### TECHNICAL FIELD

The present invention provides pharmaceutical compositions comprising Lemborexant or a pharmaceutically acceptable salt thereof and a norepinephrine reuptake inhibitor (NRI) and methods of treating Sleep Apnea comprising administering Lemborexant or a pharmaceutically acceptable salt thereof and an NRI.

### BACKGROUND

Obstructive Sleep Apnea (OSA) is a common disorder caused by collapse of the pharyngeal airway during sleep. OSA can have serious health consequences.

### SUMMARY

One aspect of the present invention provides a method of treating a subject having a condition associated with pharyngeal airway collapse, the method comprising administering to a subject in need thereof an effective amount of (i) a norepinephrine reuptake inhibitor (NRI) and (ii) Lemborexant or a pharmaceutically acceptable salt thereof. Optionally, the method may further comprise administering (iii) a muscarinic receptor antagonist (MRA).

Embodiments of this aspect of the invention may include one or more of the following optional features. In some embodiments, the NRI is a norepinephrine selective reuptake inhibitor (NSRI). In some embodiments, the NSRI is selected from the group consisting of Amedalin, Atomoxetine, CP-39,332, Daledalin, Edivoxetine, Esreboxetine, Lortalamine, Nisoxetine, Reboxetine, Talopram, Talsupram, Tandamine, and Viloxazine, or pharmaceutically acceptable salts thereof. In some embodiments, the NRI is a norepinephrine non-selective reuptake inhibitor (NNRI), e.g., selected from the group consisting of Amitriptiline, Amoxapine, Bupropion, Ciclazindol, Desipramine, Desvenlafaxine, Dexmethilphenidate, Diethylpropion, Doxepin, Duloxetine, Imipramine, Levomilnacipran, Manifaxine, Maprotiline, Methylphenidate, Milnacipran, Nefazodone, Nortriptyline, Phendimetrazine, Protryptyline, Radafaxine, Tapentadol, Teniloxazine, and Venlafaxine, or pharmaceutically acceptable salts thereof. In some embodiments, the NRI is selected from the group consisting of Atomoxetine and Reboxetine, or pharmaceutically acceptable salts thereof. In some embodiments, the NRI is Atomoxetine or a pharmaceutically acceptable salt thereof. In some embodiments, the Atomoxetine or a pharmaceutically acceptable salt thereof is administered at a dosage of from about 10 mg to about 250 mg. In some embodiments, the Atomoxetine or a pharmaceutically acceptable salt thereof is administered at a dosage of from about 25 mg to about 150 mg. In some embodiments, the Lemborexant or a pharmaceutically acceptable salt thereof is administered at a dosage of from about 1 mg to about 50 mg. In some embodiments, the Lemborexant or a pharmaceutically acceptable salt thereof is administered at a dosage of from about 2 mg to about 25 mg. In some embodiments, the NRI and Lemborexant or a pharmaceutically acceptable salt thereof are each administered daily. In some embodiments, the NRI and Lemborexant or a pharmaceutically acceptable salt thereof are administered in a single composition. In some embodiments, single composition is an oral administration form. In some embodiments, the oral administration form is a syrup, pill, tablet, troche, capsule, or patch. In some embodiments, the method further comprises administering to the subject a muscarinic receptor antagonist (MRA). In some embodiments, the MRA is selected from the group consisting of Atropine, Propantheline, Bethanechol, Solifenacin, Darifenacin, Tolterodine, Fesoterodine, Trospium, and Oxybutynin, or pharmaceutically acceptable salts thereof. In some embodiments, the MRA is selected from the group consisting of Anisotropine, Benztropine, Biperiden, Clidinium, Cycrimine, Dicyclomine, Diphemanil, Diphenidol, Ethopropazine, Glycopyrrolate, Hexocyclium, Isopropamide, Mepenzolate, Methixene, Methscopolamine, Oxyphencyclimine, Oxyphenonium, Procyclidine, Scopolamine, Tridihexethyl and Trihexyphenidyl, or pharmaceutically acceptable salts thereof. In some embodiments, the MRA is Oxybutynin or a pharmaceutically acceptable salt thereof. In some embodiments, the Oxybutynin or a pharmaceutically acceptable salt thereof is administered at a dose of from about 1 mg to about 25 mg. In some embodiments, the Oxybutynin or a pharmaceutically acceptable salt thereof is administered at a dose of from about 2 mg to about 15 mg. In some embodiments, the MRA is (R)-Oxybutynin or a pharmaceutically acceptable salt thereof. In some embodiments, the (R)-Oxybutynin or a pharmaceutically acceptable salt thereof is administered at a dose of from about 1 mg to about 25 mg. In some embodiments, the (R)-Oxybutynin or a pharmaceutically acceptable salt thereof is administered at a dose of from about 2 mg to about 15 mg. In some embodiments, the MRA is administered daily. In some embodiments, the NRI, the MRA, and Lemborexant or a pharmaceutically acceptable salt thereof are administered in a single composition. In some embodiments, the single composition is an oral administration form. In some embodiments, the oral administration form is a syrup, pill, tablet, troche, capsule, or patch. In some embodiments, the condition associated with pharyngeal airway collapse is Sleep Apnea or Simple Snoring. In some embodiments, the condition associated with pharyngeal airway collapse is Obstructive Sleep Apnea (OSA). In some embodiments, the subject is in a non-fully conscious state. In some embodiments, the non-fully conscious state is sleep. In some embodiments, the method further comprises increasing the arousal threshold in the subject.

Another aspect of the invention provides a pharmaceutical composition comprising a norepinephrine reuptake inhibitor (NRI), Lemborexant or a pharmaceutically acceptable salt thereof, and a pharmaceutically acceptable carrier. Optionally, the composition may further comprise a muscarinic receptor antagonist (MRA).

Embodiments of this aspect of the invention may include one or more of the following optional features. In some embodiments, the NRI is a norepinephrine selective reuptake inhibitor (NSRI). In some embodiments, the NSRI is selected from the group consisting of Amedalin, Atomoxetine, CP-39,332, Daledalin, Edivoxetine, Esreboxetine, Lortalamine, Nisoxetine, Reboxetine, Talopram, Talsupram, Tandamine, and Viloxazine or pharmaceutically acceptable salts thereof. In some embodiments, NRI is a norepinephrine non-selective reuptake inhibitor (NNRI), e.g., selected from the group consisting of Amitriptiline, Amoxapine, Bupropion, Ciclazindol, Desipramine, Desvenlafaxine, Dexmethilphenidate, Diethylpropion, Doxepin, Duloxetine, Imipramine, Levomilnacipran, Manifaxine, Maprotiline, Methylphenidate, Milnacipran, Nefazodone, Nortriptyline, Phendimetrazine, Protryptyline, Radafaxine, Tapentadol, Teniloxazine, and Venlafaxine, or pharmaceutically acceptable salts thereof. In some embodiments, the NRI is selected from the group consisting of Atomoxetine and Reboxetine, or pharmaceutically acceptable salts thereof. In some embodiments, the NRI is Atomoxetine or a pharmaceutically acceptable salt thereof. In some embodiments, the Atomoxetine or a pharmaceutically acceptable salt thereof is present in an amount of from about 10 mg to about 250 mg. In some embodiments, the Atomoxetine or a pharmaceutically acceptable salt thereof is present in an amount of from about 25 mg to about 150 mg. In some embodiments, the Lemborexant or a pharmaceutically acceptable salt thereof is present in an amount of from about 1 mg to about 50 mg. In some embodiments, the Lemborexant or a pharmaceutically acceptable salt thereof is present in an amount of from about 2 mg to about 25 mg. In some embodiments, the composition further comprises a muscarinic receptor antagonist (MRA). In some embodiments, the MRA is selected from the group consisting of Atropine, Propantheline, Bethanechol, Solifenacin, Darifenacin, Tolterodine, Fesoterodine, Trospium, and Oxybutynin, or pharmaceutically acceptable salts thereof. In some embodiments, the MRA is selected from the group consisting of Anisotropine, Benztropine, Biperiden, Clidinium, Cycrimine, Dicyclomine, Diphemanil, Diphenidol, Ethopropazine, Glycopyrrolate, Hexocyclium, Isopropamide, Mepenzolate, Methixene, Methscopolamine, Oxyphencyclimine, Oxyphenonium, Procyclidine, Scopolamine, Tridihexethyl and Trihexyphenidyl, or pharmaceutically acceptable salts thereof. In some embodiments, the MRA is Oxybutynin or a pharmaceutically acceptable salt thereof. In some embodiments, the Oxybutynin or a pharmaceutically acceptable salt thereof is present in amount of from about 1 mg to about 25 mg. In some embodiments, the Oxybutynin or a pharmaceutically acceptable salt thereof is present in amount of from about 2 mg to about 15 mg. In some embodiments, the MRA is (R)-Oxybutynin or a pharmaceutically acceptable salt thereof. In some embodiments, the (R)-Oxybutynin or a pharmaceutically acceptable salt thereof is present in amount of from about 1 mg to about 25 mg. In some embodiments, the (R)-Oxybutynin or a pharmaceutically acceptable salt thereof is present in amount of from about 2 mg to about 15 mg. In some embodiments, the composition is an oral administration form. In some embodiments, the oral administration form is a syrup, pill, tablet, troche, capsule, or patch. In some embodiments, the composition is for use in treating a subject having a condition associated with pharyngeal airway collapse. In some embodiments, the condition associated with pharyngeal airway collapse is Sleep Apnea or Simple Snoring. In some embodiments, the condition associated with pharyngeal airway collapse is Obstructive Sleep Apnea (OSA). In some embodiments, the subject is in a non-fully conscious state. In some embodiments, the non-fully conscious state is sleep. In some embodiments, use of the composition increases the arousal threshold in the subject.

Another aspect of the invention provides a norepinephrine reuptake inhibitor (NRI) and Lemborexant or a pharmaceutically acceptable salt thereof, and optionally a muscarinic receptor antagonist (MRA), for use in treating a subject having a condition associated with pharyngeal airway collapse.

Another aspect of the invention provides a kit comprising a norepinephrine reuptake inhibitor (NRI) and Lemborexant or a pharmaceutically acceptable salt thereof, and optionally a muscarinic receptor antagonist (MRA). In some embodiments, the kit is for use in treating a subject having a condition associated with pharyngeal airway collapse.

Unless otherwise defined, all technical and scientific terms used herein have the same meaning as commonly understood by one of ordinary skill in the art to which this invention belongs. Methods and materials are described herein for use in the present invention; other suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

The following figures are provided by way of example and are not intended to limit the scope of the claimed invention.

FIG. 1 is a graphic illustration of an obstructive apnea.

FIG. 2 is an overview of the study design of the crossover study of Example 1.

FIG. 3 is a chart showing the average hypoxic burden (HB) in patients at baseline, after placebo, and after treatment with a combination of atomoxetine and lemborexant, according to the study of Example 2.

FIG. 4 is a chart showing the average total sleep time (TST) in patients at baseline, after placebo, and after treatment with a combination of atomoxetine and lemborexant, according to the study of Example 2.

### DETAILED DESCRIPTION

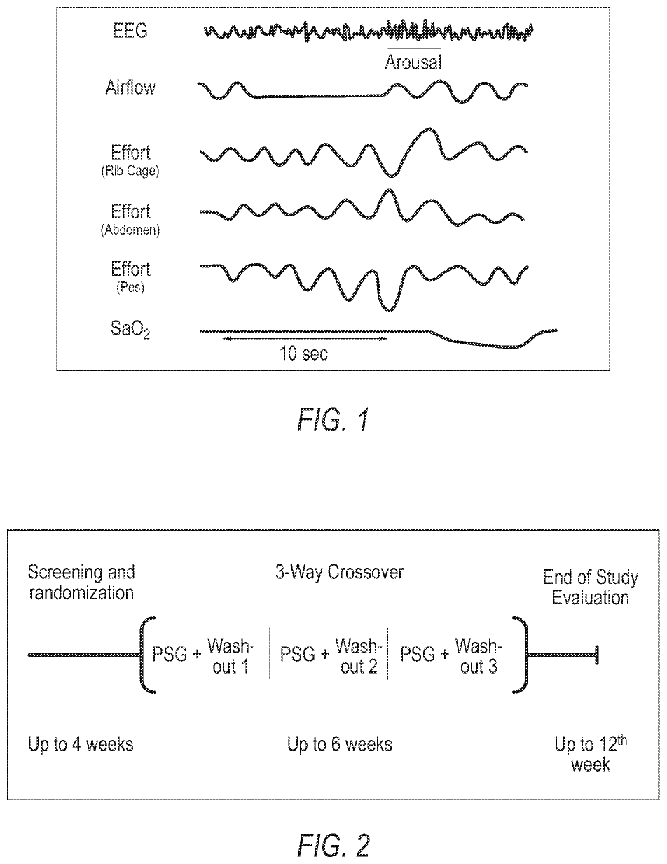

In humans, the pharyngeal airway region has no bone or cartilage support, and it is held open by muscles. When these muscles relax during sleep, the pharynx can collapse resulting in cessation of airflow. As shown in FIG. 1, ventilatory effort continues and increases in an attempt to overcome the obstruction, shown by an increase in esophageal pressure change. Rib cage and abdominal movements are in the opposite direction as a result of the diaphragm contracting against an occluded airway, forcing the abdominal wall to distend out and the chest wall to cave inward.

Increasing efforts to breathe lead to an arousal from sleep, visualisable on an EEG (FIG. 1), and result in opening of the airway and a resumption of normal breathing. The lack of airflow during the apnea also causes hypoxia, shown by a drop in oxyhemoglobin saturation (FIG. 1). Severity is generally measured using the apnea-hypopnea index (AHI), which is the combined average number of apneas (cessation of breathing for at least ten seconds) and hypopneas (reduced airflow and oxygen saturation) that occur per hour of sleep (Ruehland et al., The new AASM criteria for scoring hypopneas: Impact on the apnea hypopnea index. SLEEP 2009; 32(2): 150-157).

FIG. 1 is a graphic illustration of an obstructive apnea. The top channel shows the electroencephalogram (EEG) pattern of sleep. The next channel represents airflow. The next three channels show ventilatory effort by movements of the rib cage and abdomen and changes in esophageal pressure, all of which reflect contraction of respiratory muscles. The last channel indicates oxyhemoglobin saturation.

When a stringent definition of OSA is used (an AHI of >15 events per hour or AHI>5 events per hour with daytime sleepiness), the estimated prevalence is approximately 15 percent in males and 5 percent in females. An estimated 30 million individuals in the United States have OSA, of which approximately 6 million have been diagnosed. The prevalence of OSA in the United States appears to be increasing due to aging and increasing rates of obesity. OSA is associated with major comorbidities and economic costs, including: hypertension, diabetes, cardiovascular disease, motor vehicle accidents, workplace accidents, and fatigue/lost productivity. (Young et al., WMJ 2009; 108:246; Peppard et al., Am J Epidemiol 2013; 177:1006.)

The present leading treatment is continuous positive airway pressure (CPAP). CPAP is effective in virtually all patients, and approximately 85% of diagnosed patients are prescribed CPAP, but compliance is low. Patients find CPAP uncomfortable and often intolerable; at least 30% of patients (up to 80%) are regularly non-adherent and thus untreated (Weaver, Proc Am Thorac Soc. 2008 Feb. 15; 5(2): 173-178). Other treatment modalities with variable rates of success include oral appliances (10%) and surgery (5%), but neither is likely to be effective across the general population. No pharmacologic treatments have been shown to be effective to date.

The search for medicines to activate pharyngeal muscles in sleeping humans has been discouraging; agents such as serotonin reuptake inhibitors, tricyclic antidepressants, and sedatives have all been tested in humans and shown to be ineffective at reducing OSA severity. See, e.g., Proia and Hudgel, Chest. 1991 August; 100(2):416-21; Brownell et al., N Engl J Med 1982, 307:1037-1042; Sangal et al., Sleep Med. 2008 July; 9(5):506-10. Epub 2007 Sep. 27; Marshall et al. p. 2008 June; 31(6):824-31; Eckert et al., Clin Sci (Lond). 2011 June; 120(12); 505-14; Taranto-Montemurro et al., Sleep. 2017 Feb. 1; 40(2).

A number of pathogenic factors are thought to contribute to the development of obstructive sleep apnea (OSA) including: 1) an anatomically small, collapsible upper airway; 2) an oversensitive respiratory control system leading to ventilatory overshoots and undershoots; 3) a loss of pharyngeal muscle tone or responsiveness during sleep; and 4) a low respiratory arousal threshold, i.e., premature arousal to respiratory stimuli. The combination drug composed of atomoxetine and oxybutynin improves OSA by increasing pharyngeal muscle tone and responsiveness during sleep. This combination does not, however, improve low arousal threshold.

Methods of Treatment

The methods described herein include methods for the treatment of disorders associated with pharyngeal airway muscle collapse during sleep. In some embodiments, the disorder is Obstructive Sleep Apnea (OSA) or Simple Snoring. Generally, the methods include administering a therapeutically effective amount of a norepinephrine reuptake inhibitor (NRI) and Lemborexant or a pharmaceutically acceptable salt thereof as known in the art and/or described herein, to a subject who is in need of, or who has been determined to be in need of, such treatment. The methods optionally further include administering a muscarinic receptor antagonist (MRA). In some embodiments, the methods include administering a therapeutically effective amount of Atomoxetine or a pharmaceutically acceptable salt thereof and Lemborexant or a pharmaceutically acceptable salt thereof. The methods optionally further include administering oxybutynin (e.g., (R)-oxybutynin) or a pharmaceutically acceptable salt thereof.

As used in this context, to “treat” means to ameliorate at least one symptom of the disorder associated with pharyngeal airway collapse. Often, pharyngeal airway collapse during sleep results in snoring and/or an interruption in breathing (apnea or hypopnea), arousal from sleep, and reduced oxygenation (hypoxemia); thus, a treatment can result in a reduction in snoring, apneas/hypopneas, sleep fragmentation, and hypoxemia. Administration of a therapeutically effective amount of a compound described herein for the treatment of a subject with OSA may result in decreased AHI. Administration of a therapeutically effective amount of a compound described herein for the treatment of a subject with OSA may result in increased arousal threshold. Measurement of OSA disease and symptoms may be, for example, by polysomnography (PSG).

In general, an “effective amount” of a compound refers to an amount sufficient to elicit the desired biological response, e.g., to treat a condition associated with pharyngeal airway collapse, e.g., to treat sleep apnea or snoring. As will be appreciated by those of ordinary skill in this art, the effective amount of a compound of the invention may vary depending on such factors as the desired biological endpoint, the pharmacokinetics of the compound, the disease being treated, the mode of administration, and the age, weight, health, and condition of the subject. An effective amount encompasses therapeutic and prophylactic treatment.

An effective amount can be administered in one or more administrations, applications or dosages. The compositions can be administered from one or more times per day to one or more times per week; including once every other day. In some embodiments, the compositions are administered daily. In some embodiments, the compositions are administered daily before sleep time, e.g., immediately before sleep time or 15-60 minutes before sleep time. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, the terms “subject” and “patient” are used interchangeably. The terms “subject” and “patient” refer to an animal (e.g., a bird such as a chicken, quail or turkey, or a mammal), specifically a “mammal” including a non-primate (e.g., a cow, pig, horse, sheep, rabbit, guinea pig, rat, cat, dog, and mouse) and a primate (e.g., a monkey, chimpanzee and a human), and more specifically a human. In one embodiment, the subject is a non-human animal such as a farm animal (e.g., a horse, cow, pig or sheep), or a pet (e.g., a dog, cat, guinea pig or rabbit). In a preferred embodiment, the subject is a human.

As used herein, “pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salts” includes “pharmaceutically acceptable acid addition salts” and “pharmaceutically acceptable base addition salts.” “Pharmaceutically acceptable acid addition salts” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid, and the like, as well as organic acids such as acetic acid, trifluoroacetic acid, propionic acid, glycolic acid, pyruvic acid, oxalic acid, maleic acid, malic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

“Pharmaceutically acceptable base addition salts” include those derived from inorganic bases such as sodium, potassium, lithium, ammonium, calcium, magnesium, iron, zinc, copper, manganese, aluminum salts, and the like. Exemplary salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines, and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. (See, for example, Berge, S M. et al., “Pharmaceutical Salts,” J. Pharm. Sci., 1977; 66:1-19 which is incorporated herein by reference.)

As used herein, the term “unit dosage form” is defined to refer to the form in which the compound is administered to a subject. Specifically, the unit dosage form can be, for example, a pill, capsule, or tablet. In some embodiments, the unit dosage form is a capsule. In some embodiments, the unit dosage form is a tablet.

As used herein, “solid dosage form” means a pharmaceutical dose(s) in solid form, e.g. tablets, capsules, granules, powders, sachets, reconstitutable powders, dry powder inhalers and chewables.

For the compounds disclosed herein, single stereochemical isomers, as well as enantiomers, diastereomers, cis/trans conformation isomers, and rotational isomers, and racemic and non-racemic mixtures thereof, are within the scope of the invention. Unless otherwise indicated, all tautomeric forms of the compounds disclosed herein are within the scope of the invention.

Atomoxetine is the generic name of the pharmaceutical substance with the chemical name (−)-N-Methyl-3-phenyl-3-(o-tolyloxy)-propylamine, and its pharmaceutical salts. Atomoxetine is the R(−)-isomer as determined by x-ray diffraction. In some embodiments, atomoxetine may be atomoxetine hydrochloride.

Oxybutynin is the generic name for the pharmaceutical substance with the chemical name 4-diethylamino-2-butynylphenylcyclohexylglycolate or 4-(diethylamino)but-2-ynyl 2-cyclohexyl-2-hydroxy-2-phenylacetate, and its pharmaceutically acceptable salts. In various embodiments, oxybutynin may be a racemic mixture of R- and S-enantiomers, or an isolated enantiomer, e.g., the R-enantiomer. In various embodiments, oxybutynin may be oxybutynin chloride or (R)-oxybutynin chloride.

Lemborexant is the generic name of the pharmaceutical substance with the chemical name (1R,2S)-2-{[(2,4-dimethylpyrimidin-5-yl)oxy]methyl}-2-(3-fluorophenyl)-N-(5-fluoropyridin-2-yl) cyclopropanecarboxamide, and its pharmaceutically acceptable salts. Lemborexant is a dual orexin receptor antagonist that has been used in the treatment of insomnia.

In some embodiments, the methods include administering a dose of from about 10 mg to about 250 mg atomoxetine or a pharmaceutically acceptable salt thereof (or a dose equivalent thereof of another NRI), or in some embodiments from about 25 mg to about 150 mg atomoxetine or a pharmaceutically acceptable salt thereof. In some embodiments, the dose of atomoxetine or a pharmaceutically acceptable salt thereof is from about 50 mg to about 100 mg, e.g., about 75 mg or about 80 mg. In some embodiments, the methods include administering a dose of from about 1 mg to about 50 mg of Lemborexant or a pharmaceutically acceptable salt thereof, or in some embodiments from about 2 mg to about 25 mg Lemborexant or a pharmaceutically acceptable salt thereof. In some embodiments, the dose of Lemborexant or a pharmaceutically acceptable salt thereof from about 5 mg to about 20 mg, e.g., about 10 mg. In methods comprising administration of oxybutynin or a pharmaceutically acceptable salt thereof or (R)-oxybutynin or a pharmaceutically acceptable salt thereof (or another MRA), the dose of oxybutynin or a pharmaceutically acceptable salt thereof or (R)-oxybutynin or a pharmaceutically acceptable salt thereof may be from about 1 mg to about 25 mg (or a dose equivalent thereof of another MRA), or in some embodiments, from about 2 mg to about 15 mg. In some embodiments, the dose of oxybutynin or a pharmaceutically acceptable salt thereof is from about 2.5 mg to about 10 mg, e.g., 5 mg. In some embodiments, the dose of (R)-oxybutynin or a pharmaceutically acceptable salt thereof is from about 1 mg to about 10 mg, e.g., 2.5 mg. In some embodiments, the dose of oxybutynin or a pharmaceutically acceptable salt thereof or (R)-oxybutynin or a pharmaceutically acceptable salt thereof is from about 1 mg to about 5 mg.

In some embodiments, the methods include administering 80 mg atomoxetine or a pharmaceutically acceptable salt thereof and 10 mg Lemborexant or a pharmaceutically acceptable salt thereof. In some embodiments, the methods include administering 80 mg atomoxetine or a pharmaceutically acceptable salt thereof, 10 mg Lemborexant or a pharmaceutically acceptable salt thereof, and 5 mg oxybutynin or a pharmaceutically acceptable salt thereof. In some embodiments, the methods include administering 80 mg atomoxetine or a pharmaceutically acceptable salt thereof, 10 mg Lemborexant or a pharmaceutically acceptable salt thereof, and 2.5 mg (R)-oxybutynin or a pharmaceutically acceptable salt thereof

Pharmaceutical Compositions

Also provided herein are pharmaceutical compositions comprising a norepinephrine reuptake inhibitor (NRI) and Lemborexant or a pharmaceutically acceptable salt thereof as active ingredients. The NRI and Lemborexant or a pharmaceutically acceptable salt thereof can be in a single composition or in separate compositions. Also provided herein are pharmaceutical compositions comprising a norepinephrine reuptake inhibitor (NRI), Lemborexant or a pharmaceutically acceptable salt thereof, and a muscarinic receptor antagonist (MRA) as active ingredients. The compositions comprising an NRI, Lemborexant or a pharmaceutically acceptable salt thereof and an MRA can be in a single composition or in separate compositions.

Exemplary norepinephrine reuptake inhibitors (NRIs) include the selective NRIs including Amedalin (UK-3540-1), Atomoxetine (Strattera), CP-39,332, Daledalin (UK-3557-15), Edivoxetine (LY-2216684), Esreboxetine, Lortalamine (LM-1404), Nisoxetine (LY-94,939), Reboxetine (Edronax, Vestra), Talopram (Lu 3-010), Talsupram (Lu 5-005), Tandamine (AY-23,946), Viloxazine (Vivalan), and the non-selective NRIs including Amitriptiline, Amoxapine, Bupropion, Ciclazindol, Desipramine, Desvenlafaxine, Dexmethilphenidate, Diethylpropion, Doxepin, Duloxetine, Imipramine, Levomilnacipran, Manifaxine (GW-320,659), Maprotiline, Methylphenidate, Milnacipran, Nefazodone, Nortriptyline, Phendimetrazine, Phenmetrazine, Protryptyline, Radafaxine (GW-353,162), Tapentadol (Nucynta), Teniloxazine (Lucelan, Metatone) and Venlafaxine, or pharmaceutically acceptable salts thereof.

Exemplary muscarinic receptor antagonists (MRAs) include Atropine, Propantheline, Bethanechol, Solifenacin, Darifenacin, Tolterodine, Fesoterodine, Trospium, and Oxybutynin, or pharmaceutically acceptable salts thereof, which have activity on the M2 receptor. Other exemplary antimuscarinics include Anisotropine, Benztropine, Biperiden, Clidinium, Cycrimine, Dicyclomine, Diphemanil, Diphenidol, Ethopropazine, Glycopyrrolate, Hexocyclium, Isopropamide, Mepenzolate, Methixene, Methscopolamine, Oxyphencyclimine, Oxyphenonium, Procyclidine, Scopolamine, Tridihexethyl, and Trihexyphenidyl, or pharmaceutically acceptable salts thereof.

In some embodiments, the norepinephrine reuptake inhibitor is Atomoxetine or a pharmaceutically acceptable salt thereof.

In some embodiments, the muscarinic receptor antagonist is Oxybutynin or a pharmaceutically acceptable salt thereof or (R)-Oxybutynin or a pharmaceutically acceptable salt thereof.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

The active pharmaceutical ingredients (APIs) for use in the present invention may be provided as pharmaceutically acceptable salts. For example, Atomoxetine is commercially available as Atomoxetine hydrochloride and Oxybutynin is commercially available as Oxybutynin chloride. The APIs may alternatively be provided as a free base or free acid, e.g., Lemborexant base.

In some embodiments, patients having OSA have a low arousal threshold, which can be exacerbated by the administered norepinephrine inhibitor. In such embodiments where patients have a low arousal threshold caused or worsened by the use of one or more norepinephrine inhibitors (e.g., Atomoxetine), Lemborexant may increase the arousal threshold of the patient having OSA, pharyngeal airway collapse, or a combination thereof. In some embodiments, the arousal threshold of a patient can be measured by polysomnography (PSG).

Pharmaceutical compositions are typically formulated to be compatible with its intended route of administration. Examples of routes of administration include systemic oral or transdermal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, oral compositions generally include an inert diluent or an edible carrier. For the purpose of oral therapeutic administration, the active compound(s) can be incorporated with excipients and used in the form of pills, tablets, troches, or capsules, e.g., gelatin capsules. Oral compositions can also be prepared using a fluid carrier. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration of the compounds as described herein can also be by transdermal means, e.g., using a patch, gel, or lotion, to be applied to the skin. For transdermal administration, penetrants appropriate to the permeation of the epidermal barrier can be used in the formulation. Such penetrants are generally known in the art. For example, for transdermal administration, the active compounds can formulated into ointments, salves, gels, or creams as generally known in the art. The gel and/or lotion can be provided in individual sachets, or via a metered-dose pump that is applied daily; see, e.g., Cohn et al., Ther Adv Urol. 2016 April; 8(2): 83-90.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration or use in a method described herein.

### Examples

The invention is further described in the following examples, which do not limit the scope of the invention described in the claims.

### Example 1. A Phase 2 Randomized, Double Blind, Placebo-Controlled, Single-Dose, 3-Period Crossover Study to Evaluate the Efficacy of Combinations of Atomoxetine, Oxybutynin, and Lemborexant in Obstructive Sleep Apnea

A placebo-controlled, double-blinded, randomized, crossover trial in OSA human patients is performed. Participants receive double combination treatment (atomoxetine hydrochloride capsules 80 mg+lemborexant tablets 10 mg), triple combination treatment (atomoxetine hydrochloride capsules 80 mg+lemborexant tablets 10 mg+oxybutynin chloride tablets 5 mg) or placebo in randomized order immediately before sleep. The combination of atomoxetine and lemborexant and the triple combination with oxybutynin is tested for its ability to reduce the apnea hypopnea index, increase the arousal threshold, and improve OSA severity. The drug combinations are also tested for ability to increase genioglossus muscle responsiveness to an increase in ventilatory drive, improve upper airway muscle activity, improve ventilation, increase oxygen levels (SaO2), increase total sleep time and improve sleep efficiency.

An overview of the study design is shown in FIG. 2. A four-week screening and randomization period is followed by a three-way crossover period of up to six weeks. Participants remaining in the study after screening receive a screening polysomnography (PSG). Participants then receive double combination treatment, triple combination treatment, or placebo during the crossover period. The three-way crossover period includes three separate PSG measurements, each followed by a 1-week washout period.

### Example 2. Clinical Study

The aim of this study was to determine the effect of the combination of atomoxetine and lemborexant on OSA severity and sleep.

After a baseline PSG, 15 patients completed a randomized, placebo-controlled, crossover trial comparing 1 night of 80 mg atomoxetine plus 10 mg lemborexant to placebo. Patients with previously identified features of low-collapsible upper airway were included in the trial (average desaturation linked to obstructive events <8%, higher number of hypopneas compared to apneas). AHI was calculated using 4% desaturation definition for hypopneas. Sleep apnea specific hypoxic burden (HB) was calculated as the area under the saturation curve following an obstructive event.

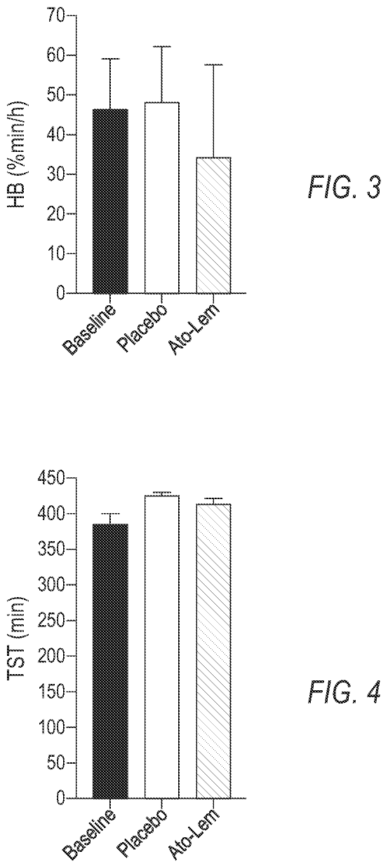

Results of the trial for hypoxic burden (HB) and total sleep time (TST) are shown in FIGS. 3 and 4, respectively. Hypoxic burden was lower in the subjects receiving the atomoxetine plus lemborexant combination treatment compared to baseline and placebo.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating a subject having a condition associated with pharyngeal airway collapse, the method comprising administering to a subject in need thereof an effective amount of (i) a norepinephrine reuptake inhibitor (NRI) and (ii) Lemborexant or a pharmaceutically acceptable salt thereof.

2. The method of claim 1, wherein the NRI is a norepinephrine selective reuptake inhibitor (NSRI).

3. The method of claim 2, wherein the NSRI is selected from the group consisting of Amedalin, Atomoxetine, CP-39,332, Daledalin, Edivoxetine, Esreboxetine, Lortalamine, Nisoxetine, Reboxetine, Talopram, Talsupram, Tandamine, and Viloxazine, or pharmaceutically acceptable salts thereof.

4. The method of claim 1, wherein the NRI is a norepinephrine non-selective reuptake inhibitor (NNRI) selected from the group consisting of Amitriptiline, Amoxapine, Bupropion, Ciclazindol, Desipramine, Desvenlafaxine, Dexmethilphenidate, Diethylpropion, Doxepin, Duloxetine, Imipramine, Levomilnacipran, Manifaxine, Maprotiline, Methylphenidate, Milnacipran, Nefazodone, Nortriptyline, Phendimetrazine, Protryptyline, Radafaxine, Tapentadol, Teniloxazine, and Venlafaxine, or pharmaceutically acceptable salts thereof.

5. The method of claim 1, wherein the NRI is selected from the group consisting of Atomoxetine and Reboxetine, or pharmaceutically acceptable salts thereof.

6. The method of claim 5, wherein the NRI is Atomoxetine or a pharmaceutically acceptable salt thereof.

7. The method of claim 6, wherein the Atomoxetine or a pharmaceutically acceptable salt thereof is administered at a dosage of from about 10 mg to about 250 mg.

8. The method of claim 6, wherein the Atomoxetine or a pharmaceutically acceptable salt thereof is administered at a dosage of from about 25 mg to about 150 mg.

9. The method of claim 1, wherein the Lemborexant or a pharmaceutically acceptable salt thereof is administered at a dosage of from about 1 mg to about 50 mg.

10. The method of claim 1, wherein the Lemborexant or a pharmaceutically acceptable salt thereof is administered at a dosage of from about 2 mg to about 25 mg.

11. The method of any one of claims 1-10, wherein the NRI and Lemborexant or a pharmaceutically acceptable salt thereof are each administered daily.

12. The method of any one of claims 1-11, wherein the NRI and Lemborexant or a pharmaceutically acceptable salt thereof are administered in a single composition.

13. The method of claim 12, wherein the single composition is an oral administration form.

14. The method of claim 13, wherein the oral administration form is a syrup, pill, tablet, troche, capsule, or patch.

15. The method of any one of claims 1-14, further comprising administering to the subject a muscarinic receptor antagonist (MRA).

16. The method of claim 15, wherein the MRA is selected from the group consisting of Atropine, Propantheline, Bethanechol, Solifenacin, Darifenacin, Tolterodine, Fesoterodine, Trospium, and Oxybutynin, or pharmaceutically acceptable salts thereof.

17. The method of claim 15, wherein the MRA is selected from the group consisting of Anisotropine, Benztropine, Biperiden, Clidinium, Cycrimine, Dicyclomine, Diphemanil, Diphenidol, Ethopropazine, Glycopyrrolate, Hexocyclium, Isopropamide, Mepenzolate, Methixene, Methscopolamine, Oxyphencyclimine, Oxyphenonium, Procyclidine, Scopolamine, Tridihexethyl and Trihexyphenidyl, or pharmaceutically acceptable salts thereof.

18. The method of claim 15, wherein the MRA is Oxybutynin or a pharmaceutically acceptable salt thereof.

19. The method of claim 15, wherein the MRA is (R)-Oxybutynin or a pharmaceutically acceptable salt thereof.

20. The method of claim 18, wherein the Oxybutynin or a pharmaceutically acceptable salt thereof is administered at a dose of from about 1 mg to about 25 mg.

21. The method of claim 18, wherein the Oxybutynin or a pharmaceutically acceptable salt thereof is administered at a dose of from about 2 mg to about 15 mg.

22. The method of claim 19, wherein the (R)-Oxybutynin or a pharmaceutically acceptable salt thereof is administered at a dose of from about 1 mg to about 25 mg.

23. The method of claim 19, wherein the (R)-Oxybutynin or a pharmaceutically acceptable salt thereof is administered at a dose of from about 2 mg to about 15 mg.

24. The method of any one of claims 15-23, wherein the MRA is administered daily.

25. The method of any one of claims 15-24, wherein the NRI, the MRA, and Lemborexant or a pharmaceutically acceptable salt thereof are administered in a single composition.

26. The method of claim 25, wherein the single composition is an oral administration form.

27. The method of claim 26, wherein the oral administration form is a syrup, pill, tablet, troche, capsule, or patch.

28. The method of any one of claims 1-27, wherein the condition associated with pharyngeal airway collapse is Sleep Apnea or Simple Snoring.

29. The method of claim 28, wherein the condition associated with pharyngeal airway collapse is Obstructive Sleep Apnea (OSA).

30. The method of any one of claims 1-29, wherein the subject is in a non-fully conscious state.

31. The method of claim 30, wherein the non-fully conscious state is sleep.

32. The method of any one of claims 1-31, comprising increasing the arousal threshold in the subject.

33. A pharmaceutical composition comprising a norepinephrine reuptake inhibitor (NRI), Lemborexant or a pharmaceutically acceptable salt thereof, and a pharmaceutically acceptable carrier.

34. The composition of claim 33, wherein the NRI is a norepinephrine selective reuptake inhibitor (NSRI).

35. The composition of claim 34, wherein the NSRI is selected from the group consisting of Amedalin, Atomoxetine, CP-39,332, Daledalin, Edivoxetine, Esreboxetine, Lortalamine, Nisoxetine, Reboxetine, Talopram, Talsupram, Tandamine, and Viloxazine, or pharmaceutically acceptable salts thereof.

36. The composition of claim 33, wherein the NRI is a norepinephrine non-selective reuptake inhibitor (NNRI) selected from the group consisting of Amitriptiline, Amoxapine, Bupropion, Ciclazindol, Desipramine, Desvenlafaxine, Dexmethilphenidate, Diethylpropion, Doxepin, Duloxetine, Imipramine, Levomilnacipran, Manifaxine, Maprotiline, Methylphenidate, Milnacipran, Nefazodone, Nortriptyline, Phendimetrazine, Protryptyline, Radafaxine, Tapentadol, Teniloxazine, and Venlafaxine, or pharmaceutically acceptable salts thereof.

37. The composition of claim 33, wherein the NRI is selected from the group consisting of Atomoxetine and Reboxetine, or pharmaceutically acceptable salts thereof.

38. The composition of claim 37, wherein the NRI is Atomoxetine or a pharmaceutically acceptable salt thereof.

39. The composition of claim 38, wherein the Atomoxetine or a pharmaceutically acceptable salt thereof is present in an amount of from about 10 mg to about 250 mg.

40. The composition of claim 38, wherein the Atomoxetine or a pharmaceutically acceptable salt thereof is present in an amount of from about 25 mg to about 150 mg.

41. The composition of claim 33, wherein the Lemborexant or a pharmaceutically acceptable salt thereof is present in an amount of from about 1 mg to about 50 mg.

42. The composition of claim 33, wherein the Lemborexant or a pharmaceutically acceptable salt thereof is present in an amount of from about 2 mg to about 25 mg.

43. The composition of any one of claims 33-42, further comprising a muscarinic receptor antagonist (MRA).

44. The composition of claim 43, wherein the MRA is selected from the group consisting of Atropine, Propantheline, Bethanechol, Solifenacin, Darifenacin, Tolterodine, Fesoterodine, Trospium, and Oxybutynin, or pharmaceutically acceptable salts thereof.

45. The composition of claim 43, wherein the MRA is selected from the group consisting of Anisotropine, Benztropine, Biperiden, Clidinium, Cycrimine, Dicyclomine, Diphemanil, Diphenidol, Ethopropazine, Glycopyrrolate, Hexocyclium, Isopropamide, Mepenzolate, Methixene, Methscopolamine, Oxyphencyclimine, Oxyphenonium, Procyclidine, Scopolamine, Tridihexethyl and Trihexyphenidyl, or pharmaceutically acceptable salts thereof.

46. The composition of claim 43, wherein the MRA is Oxybutynin or a pharmaceutically acceptable salt thereof.

47. The composition of claim 43, wherein the MRA is (R)-Oxybutynin or a pharmaceutically acceptable salt thereof.

48. The composition of claim 46, wherein the Oxybutynin or a pharmaceutically acceptable salt thereof is present in amount of from about 1 mg to about 25 mg.

49. The composition of claim 46, wherein the Oxybutynin or a pharmaceutically acceptable salt thereof is present in amount of from about 2 mg to about 15 mg.

50. The composition of claim 47, wherein the (R)-Oxybutynin or a pharmaceutically acceptable salt thereof is present in amount of from about 1 mg to about 25 mg.

51. The composition of claim 47, wherein the (R)-Oxybutynin or a pharmaceutically acceptable salt thereof is present in amount of from about 2 mg to about 15 mg.

52. The composition of any one of claims 33-51, wherein the composition is an oral administration form.

53. The composition of claim 52, wherein the oral administration form is a syrup, pill, tablet, troche, capsule, or patch.

54. The composition of any one of claims 33-53, for use in treating a subject having a condition associated with pharyngeal airway collapse.

55. The composition for use of claim 54, wherein the condition associated with pharyngeal airway collapse is Sleep Apnea or Simple Snoring.

56. The composition for use of claim 55, wherein the condition associated with pharyngeal airway collapse is Obstructive Sleep Apnea (OSA).

57. The composition for use of any one of claims 54-56, wherein the subject is in a non-fully conscious state.

58. The composition for use of claim 57, wherein the non-fully conscious state is sleep.

59. The composition for use of any one of claims 54-58, wherein the composition increases the arousal threshold in the subject.

60. A norepinephrine reuptake inhibitor (NRI) and Lemborexant or a pharmaceutically acceptable salt thereof, and optionally a muscarinic receptor antagonist (MRA), for use in treating a subject having a condition associated with pharyngeal airway collapse.

61. A kit comprising a norepinephrine reuptake inhibitor (NRI) and Lemborexant or a pharmaceutically acceptable salt thereof, and optionally a muscarinic receptor antagonist (MRA).

62. The kit of claim 61, for use in treating a subject having a condition associated with pharyngeal airway collapse.
